SEQUENCE LISTING (25 sequences)

SEQ ID NO: 1 - protein, 218 aa, Medicago truncatula
MEQTQQEIGIKIYNTTPPPQEAMGAVTQQPSDPPEHGKKRRAIMAKGVQKTLSKTSLLGNFLPSGTLLTFEMVLPSIYRNGQCTHVHTIMIHFLLIICALSCFFFHFTDSFHGADGNVYYGFVTPKGLSVFKPGLAVLVPNDDKYKVGFQDFIHAVMSVMVFVAIAFSDYRVSNCLFPGHEREMDQVMESFPLMVGIVCSGLFLIFPTSRRGIGCMSA

SEQ ID NO: 2 - protein, 218 aa, Medicago truncatula
MEQTQQEIGIKVYNATPPPQEAIGGVTHQPSDPPEPGKKRRAIMAKGVQKTLSKTSLLGNFLPTGTLITFEMVLPSIYRNGQCTHVHTIMIHFLLIMCALSCFFFHFTDSFHGADGNVYYGFATRNGLSVFKPGLTVLVPNDDKYKVGFQDFVHAVMSVMVFVAIAFSDYRVTNCLFPGHEKEMDQVMESFPLMVGIICSGLFLIFPTSRHGIGCMSS

SEQ ID NO: 3 - DNA, 657 bp, Medicago truncatula
  source 1..657: mol_type = other DNA
atggaacaaacacaacaagaaattggaatcaaaatctacaacacaacaccgccaccacaagaagccatgggggccgtaacgcagcagccttctgatccaccggaacatggcaagaagcgtcgtgccataatggcgaaaggcgtgcaaaaaaccctttcaaaaacttccttacttggtaacttccttccatcaggaacactcctcacattcgaaatggtccttccttcgatctataggaacggccaatgcactcacgtacacaccatcatgatccatttccttttaattatatgtgcactctcttgtttcttttttcactttacagatagttttcacggcgctgatggtaacgtttactacggttttgttaccccgaaagggttatccgtttttaaaccaggacttgctgttttggttcctaacgacgacaaatacaaggtagggtttcaagattttattcatgcggttatgtcggttatggtgtttgtggcgatcgctttttcggattatagggtgagtaattgtttgtttcctggacatgaaagggaaatggatcaagttatggagagttttccattgatggttggaattgtttgtagcggtttgtttcttatttttccaacttcaaggcgtggaattggatgcatgtctgcctaa

SEQ ID NO: 4 - DNA, 657 bp, Medicago truncatula
  source 1..657: mol_type = other DNA
atggaacaaactcaacaagaaattgggatcaaagtctacaatgcaacacctccaccacaagaggccataggcggcgtcacacaccagccttccgatccaccggaacctggcaagaaacgtcgcgccataatggcaaaaggtgtccaaaaaaccctctcaaaaacatccttactcggaaacttccttccaacaggaacactcataacattcgaaatggtccttccttcaatctaccgaaatggtcaatgcactcatgttcataccatcatgatccatttcctcttaatcatgtgtgcactctcttgtttcttctttcactttaccgatagttttcacggcgcggatggtaacgtttactatggttttgctactcggaacgggttgtcggtttttaaaccgggactcactgttttggttcctaatgatgacaagtacaaagttgggtttcaagatttcgtgcatgcggttatgtcggttatggtgtttgtggctatcgctttttcggattatagggttactaattgtttatttcctggacatgaaaaagagatggatcaagttatggagagttttcctttgatggttggaataatttgtagcggtttgttccttatttttcctacttcaaggcatggaattggatgcatgtcttcctaa

SEQ ID NO: 5 - DNA, 23 bp, synthetic construct
  source 1..23: mol_type = other DNA
gccaccacaagaagccatggggg

SEQ ID NO: 6 - DNA, 23 bp, synthetic construct
  source 1..23: mol_type = other DNA
tggccgttcctatagatcgaagg

SEQ ID NO: 7 - DNA, 23 bp, synthetic construct
  source 1..23: mol_type = other DNA
ccaccacaagaggccataggcgg

SEQ ID NO: 8 - DNA, 23 bp, synthetic construct
  source 1..23: mol_type = other DNA
taccgatagttttcacggcgcgg

SEQ ID NO: 9 - DNA, 35 bp, synthetic construct
  source 1..35: mol_type = other DNA
caaaaaagcaggcttcatggaacaaacacaacaag

SEQ ID NO: 10 - DNA, 33 bp, synthetic construct
  source 1..33: mol_type = other DNA
caagaaagctgggtcggcagacatgcatccaat

SEQ ID NO: 11 - DNA, 50 bp, synthetic construct
  source 1..50: mol_type = other DNA
ggggacaagtttgtacaaaaaagcaggcttcatggaacaaactcaacaag

SEQ ID NO: 12 - DNA, 48 bp, synthetic construct
  source 1..48: mol_type = other DNA
ggggaccactttgtacaagaaagctgggtcggaagacatgcatccaat

SEQ ID NO: 13 - DNA, 33 bp, synthetic construct
  source 1..33: mol_type = other DNA
gtggggacaagtttgtacaaaaaagcaggcttc

SEQ ID NO: 14 - DNA, 32 bp, synthetic construct
  source 1..32: mol_type = other DNA
gtggggaccactttgtacaagaaagctgggtc

SEQ ID NO: 15 - DNA, 32 bp, synthetic construct
  source 1..32: mol_type = other DNA
tgctcttcgcgctggcagacatactgtcccac

SEQ ID NO: 16 - DNA, 43 bp, synthetic construct
  source 1..43: mol_type = other DNA
tcgtctcctcttgtggtggcctgcctatacggcagtgaacctg

SEQ ID NO: 17 - DNA, 40 bp, synthetic construct
  source 1..40: mol_type = other DNA
tcgtctcaaagaagccatgggttttagagctagaaatagc

SEQ ID NO: 18 - DNA, 40 bp, synthetic construct
  source 1..40: mol_type = other DNA
tcgtctcataggaacggccactgcctatacggcagtgaac

SEQ ID NO: 19 - DNA, 40 bp, synthetic construct
  source 1..40: mol_type = other DNA
tcgtctcacctatagatcgagttttagagctagaaatagc

SEQ ID NO: 20 - DNA, 40 bp, synthetic construct
  source 1..40: mol_type = other DNA
tcgtctcactcttgtggtggctgcctatacggcagtgaac

SEQ ID NO: 21 - DNA, 40 bp, synthetic construct
  source 1..40: mol_type = other DNA
tcgtctcaagaggccatagggttttagagctagaaatagc

SEQ ID NO: 22 - DNA, 40 bp, synthetic construct
  source 1..40: mol_type = other DNA
tcgtctcaaaactatcggtactgcctatacggcagtgaac

SEQ ID NO: 23 - DNA, 40 bp, synthetic construct
  source 1..40: mol_type = other DNA
tcgtctcagttttcacggcggttttagagctagaaatagc

SEQ ID NO: 24 - DNA, 43 bp, synthetic construct
  source 1..43: mol_type = other DNA
tcgtctcactcttgtggtggctgcctatacggcagtgaacctg

SEQ ID NO: 25 - DNA, 32 bp, synthetic construct
  source 1..32: mol_type = other DNA
tgctcttctgacctgcctatacggcagtgaac

(19) United States
(12) Patent Application Publication
(10) Pub. No.: US 2024/0254506 A1
(43) Pub. Date: Aug. 1, 2024

(54) DMP PROTEIN, ENCODING GENE AND USE THEREOF

(71) Applicant: Biotechnology Research Institute, Chinese Academy of Agricultural Sciences, Beijing (CN)

(72) Inventors: Hao LIN, Beijing (CN); Na Wang, Beijing (CN); Lifang Niu, Beijing (CN)

(21) Appl. No.: 18/640,590

(22) Filed: Apr. 19, 2024

Related U.S. Application Data
(63) Continuation of application No. PCT/CN2022/140182, filed on Dec. 20, 2022.

(30) Foreign Application Priority Data
Oct. 21, 2021 (CN) 202111225771.7

(51) Int. Cl. C12N 15/82; C12N 9/22; C12N 15/11

(52) U.S. Cl. CPC C12N 15/8287; C12N 9/22; C12N 15/11; C12N 15/8213; C12N 2310/20; C12N 2800/80

(57) ABSTRACT

Disclosed in the present invention are a DMP protein and a coding gene and use thereof. Disclosed is a complete set of proteins consisting of protein A (i.e. DMP8) and protein B (i.e. DMP9). The amino acid sequence of protein A is SEQ ID No. 1, and the amino acid sequence of protein B is SEQ ID No. 2. Also disclosed in the present invention are a method for constructing a plant haploid inducer line and a use thereof, and a plant haploid inducer line constructed by the method.

### RELATED APPLICATIONS

The present application is a Continuation of International Application Number PCT/CN2022/140182 filed Dec. 20, 2022, which claims priority to Chinese Application Number 202111225771.7, filed Oct. 21, 2021, the disclosures of which are hereby incorporated by reference herein in their entireties.

### INCORPORATION BY REFERENCE

The sequence listing provided in the file entitled C6351-123_PUS1232680_2.xml, which is an Extensible Markup Language (XML) file that was created on Apr. 17, 2024, and which comprises 24,850 bytes, is hereby incorporated by reference in its entirety.

### TECHNICAL FIELD

The present invention relates to the technical field of genetic engineering, in particular to DMP protein, encoding gene and application thereof.

### BACKGROUND OF THE INVENTION

Haploid breeding has become one of the important methods for breeding new plant varieties, while improving the haploid induction rate and simplifying the haploid induction procedure are the critical procedures for haploid breeding technology. With the development and improvement of haploid induction technology, haploid breeding technology has been widely used in the breeding research of many important crops, showing the advantages of allowing rapid homozygosity of a gene, breeding time shorten, high breeding efficiency, etc. Legume plants are important economic crops, and the in vivo haploid induction system has not been developed yet. If haploid breeding can be achieved, it will have a wide application prospect in agricultural production.

Medicago truncatula, as a model plant of Leguminosae, has the common characteristics of Leguminosae. Therefore, it is of great application value to study haploid induction mechanism of Medicago truncatula and develop an in vivo haploid induction system suitable for Leguminosae.

### SUMMARY OF THE INVENTION

The present invention aims to provide DMP proteins and encoding genes and applications thereof.

In a first aspect, the present invention claims a set of protein.

The set of proteins claimed by the present invention consists of protein A and protein B. The protein A and the protein B are both derived from Medicago truncatula, designated as DMP8 and DMP9, respectively.

The protein A (i.e., DMP8) may be any one of the followings:
(A1) a protein having an amino acid sequence of SEQ ID No. 1;
(A2) a protein obtained by substituting and/or deleting and/or adding of one or more amino acid residues to the amino acid sequence shown in SEQ ID No. 1 and having the same function;
(A3) a protein having 99% or more, 95% or more, 90% or more, 85% or more or 80% or more identity with the amino acid sequence shown in any one of (A1) to (A2) and having the same function; and
(A4) a fusion protein obtained by attaching a protein tag to the N-terminus and/or C-terminus of the protein shown in any one of (A1)-(A3).

The protein B is any one of the followings:
(B1) a protein having an amino acid sequence of SEQ ID No. 2;
(B2) a protein obtained by substituting and/or deleting and/or adding of one or more amino acid residues to the amino acid sequence shown in SEQ ID No. 2 and having the same function;
(B3) a protein having 99% or more, 95% or more, 90% or more, 85% or more or 80% or more identity with the amino acid sequence shown in any one of (B1) to (B2) and having the same function; and
(B4) a fusion protein obtained by attaching a protein tag to the N-terminus and/or C-terminus of the protein defined in any one of (B1) to (B3).

The above proteins may be synthesized artificially or obtained by synthesizing the encoding genes thereof and followed by biologically expression.

Of the above proteins, the tag refers to a polypeptide or protein that is expressed together in fusion with a target protein by using DNA in vitro recombination, so as to facilitate the expression, detection, tracing and/or purification of the target protein. The tag may be a Flag tag, His tag, MBP tag, HA tag, myc tag, GST tag and/or SUMO tag and the like.

Of the above proteins, the identity refers to the identity to an amino acid sequence. The identity to the amino acid sequence may be determined using the homology search site on the international internet, such as BLAST webpage on the NCBI homepage website. For example, the value of identity (%) may be obtained by retrieving the identity of a pair of amino acid sequences for calculation in the advanced BLAST2.1, using blast program, setting the Expect value to 10, all Filters to OFF, using BLOSUM62 as Matrix, setting Gap existence cost, Per residual gap cost, and Lambda ratio to 11, 1, and 0.85 (default values), respectively.

Of the above proteins, the identity of 99% or more may be an identity of at least 99.1%, 99.2%, 99.3%, 99.4%, 99.5%, 99.6%, 99.7%, 99.8%, 99.9%, or 100%. The identity of 95% or more may be an identity of at least 96%, 97%, 98%. The identity of 90% or more may be an identity of at least 91%, 92%, 93%, 94%. The identity of 85% or more may be an identity of at least 86%, 87%, 88%, 89%. The identity of more than 80% may be an identity of at least 81%, 82%, 83%, 84%.

In a second aspect, the present invention claims a set of nucleic acid molecules.

The set of nucleic acid molecules claimed by the present invention consists of a nucleic acid molecule A and a nucleic acid molecule B.

The nucleic acid molecule A is a nucleic acid molecule capable of expressing the protein A described above; The nucleic acid molecule B is a nucleic acid molecule capable of express the protein B described above.

The nucleic acid molecule may be DNA, such as cDNA, genomic DNA or recombinant DNA; The nucleic acid molecule may also be RNA, such as mRNA or hnRNA, etc.

Further, the nucleic acid molecule A (designated MtDMP8) may be a DNA molecule shown in any one of the followings:
(a1) a DNA molecule shown in SEQ ID No. 3;
(a2) a DNA molecule that hybridizes to the DNA molecule shown in (a1) under stringent conditions and encodes the protein A; and
(a3) a DNA molecule having 99% or more, 95% or more, 90% or more, 85% or more or 80% or more identity with the DNA sequence shown in (a1) or (a2) and encoding the protein A;
the nucleic acid molecule B (designated MtDMP9) may be a DNA molecule shown in any one of the followings:
(b1) a DNA molecule shown in SEQ ID No. 4;
(b2) a DNA molecule that hybridizes to the DNA molecule shown in (b1) under stringent conditions and encodes the protein B; and
(b3) a DNA molecule having 99% or more, 95% or more, 90% or more, 85% or more or 80% or more identity with the DNA sequence shown in (b1) or (b2) and encoding the protein B.

Of the above nucleic acid molecules, the stringent conditions may be as follows: hybridizing in a mixed solution of 7% sodium dodecyl sulfate (SDS), 0.5 M Na₃PO₄, and 1 mM EDTA at 50° C., followed by rinsing in 2×SSC and 0.1% SDS at 50° C.; alternatively, hybridizing in a mixed solution of 7% SDS, 0.5 M Na₃PO₄, and 1 mM EDTA at 50° C., followed by rinsing in 1×SSC and 0.1% SDS at 50° C.; alternatively, hybridizing in a mixed solution of 7% SDS, 0.5 M Na₃PO₄, and 1 mM EDTA at 50° C., followed by rinsing in 0.5×SSC and 0.1% SDS at 50° C.; alternatively, hybridizing in a mixed solution of 7% SDS, 0.5 M Na₃PO₄, and 1 mM EDTA at 50° C., followed by rinsing in 0.1×SSC and 0.1% SDS at 50° C.; alternatively, hybridizing in a mixed solution of 7% SDS, 0.5 M Na₃PO₄, and 1 mM EDTA at 50° C., followed by rinsing in 0.1×SSC and 0.1% SDS at 65° C.; alternatively, hybridizing in a solution of 6×SSC and 0.5% SDS at 65° C., followed by washing the membrane once each in 2×SSC and 0.1% SDS and 1×SSC and 0.1% SDS.

Of the above nucleic acid molecules, the homology refers to the identity to the nucleotide sequence. The identity to the nucleotide sequence may be determined using the homology search site on the international internet, such as BLAST webpage on the NCBI homepage website. For example, the value of identity (%) may be obtained by retrieving the identity of a pair of nucleotide sequences for calculation in the advanced BLAST2.1, using blast program, setting the Expect value to 10, all Filters to OFF, using BLOSUM62 as Matrix, and setting Gap existence cost, Per residual gap cost, and Lambda ratio to 11, 1, and 0.85 (default values), respectively.

Of the above nucleic acid molecules, the homology of 95% or more may be an identity of at least 96%, 97%, and 98%. The homology of 90% or more may be an identity of at least 91%, 92%, 93%, and 94%. The homology of 85% may be an identity of at least 86%, 87%, 88%, and 89%. The homology of 80% or more may be an identity of at least 81%, 82%, 83%, and 84%.

In a third aspect, the present invention claims any of the following biomaterials:
P1. a set of expression cassettes consisting of an expression cassette A and an expression cassette B; wherein the expression cassette A is an expression cassette containing the nucleic acid molecule A described above; the expression cassette B is an expression cassette containing the nucleic acid molecule B described above;
P2. a set of recombinant vectors, consisting of recombinant vector A and recombinant vector B; wherein the recombinant vector A is a recombinant vector containing the nucleic acid molecule A as describe above; the recombinant vector B is a recombinant vector containing the nucleic acid molecule B described above;
P3. a set of recombinant bacteria, consisting of recombinant bacteria A and B; wherein the recombinant bacterium A is a recombinant bacterium containing the nucleic acid molecule A described above; the recombinant bacterium B is a recombinant bacterium containing the nucleic acid molecule B described above;
P4. a set of transgenic cell lines, consisting of a transgenic cell line A and a transgenic cell line B; wherein the transgenic cell line A is a transgenic cell line containing the nucleic acid molecule A described above; the transgenic cell line B is a transgenic cell line containing the nucleic acid molecule B described above;
P5. a set of sgRNAs, consisting of sgRNA molecule A and sgRNA molecule B; wherein the sgRNA molecule A is an sgRNA molecule for target knockout of the nucleic acid molecule A described above; the sgRNA molecule B is an sgRNA molecule for target knockout of the nucleic acid molecule B described above;
P6. A set of CRISPR-Cas9 system (products), consisting of CRISPR-Cas9 system A and CRISPR-Cas9 system B; wherein the CRISPR-Cas9 system A consists of the sgRNA molecule A and the Cas9 protein in P5; and the CRISPR-Cas9 system B consists of the sgRNA molecule B and the Cas9 protein in P5; and
P7. CRISPR-Cas9 knockout vector containing the encoding genes of the sgRNA molecule A, the sgRNA molecule B and the Cas9 protein in P5.

In a specific embodiment of the present invention, P5-P7, the target sequences of the sgRNA molecule A are SEQ ID No. 5 and SEQ ID No. 6. The target sequences of the sgRNA molecule B are SEQ ID No. 7 and SEQ ID No. 8. The CRISPR-Cas9 knockout vectors are MtCRISPR/CaS9::MtDMP8, MtCRISPR/CaS9::MtDMP9 and/or MtCRISPR/CaS9::MtDMP8MtDMP9.

In a fourth aspect, the present invention claims the application of a set of proteins according to the first aspect described above or a set of nucleic acid molecules according to the second aspect described above or a set of biomaterials according to the third aspect described above in any of the followings:
Q1. constructing a plant haploid induction line; and
Q2. plant haploid breeding;

In the applications, the expression and/or activity of the set of proteins (i.e. the protein A and the protein B as described above) in the plant is reduced (e.g., by causing the translation of the corresponding protein to terminate prematurely) and the haploid may be obtained from offsprings of the resulting positive plants by self-crossing or hybridization.

In a fifth aspect, the present invention claims a method of constructing a plant haploid induction line.

The method of constructing a plant haploid induction line as claimed in the present invention may include the steps of: reducing the expression and/or activity of both the aforementioned protein A and the protein B in the recipient plant (e.g., by causing the translation of the corresponding protein to terminate prematurely) and the haploid may be obtained from offsprings of the resulting positive plants by self-crossing or hybridization.

Further, the method may include the steps of: depressing expression of both the nucleic acid molecule A and the nucleic acid molecule B as described above in the recipient plant to obtain a transgenic plant; and obtaining a haploid induction line from self-crossed or hybridized offsprings of the transgenic plant.

Wherein depressing expression of both the nucleic acid molecule A and the nucleic acid molecule B in the recipient plant as described hereinabove may be achieved by any technical means include, but not limited to, knockout of the nucleic acid molecule A and the nucleic acid molecule B in the recipient plant using the CRISPR-Cas9.

For example, it may be achieved by introducing into the recipient plant the set of CRISPR-Cas9 system in P6 or the CRISPR-Cas9 knockout vector in P7 (due to the insertion or deletion of nucleotides, the reading frame is shifted and the translated protein is terminated prematurely) of the previous third aspect.

The hybrid offspring is obtained by crossing the transgenic plant with other varieties of the plant.

Specifically, the method may include the steps of:
(1) Selecting target fragments according to the exon regions of MtDMP8 and MtDMP9 genes; wherein one strand in the double-stranded structure of the target fragment has an NGG structure, wherein N represents any one of bases A, T, C, and G.
(2) constructing binary expression vectors MtCRISPR/Cas9::MtDMP8, MtCRISPR/Cas9::MtDMP9 and MtCRISPR/Cas9::MtDMP8MtDMP9 for targeting of the MtDMP8 and MtDMP9 genes for Agrobacterium tumefaciens-mediated transformation according to the nucleotide arrangement order of the target sequences, and the MtCRISPR/Cas9 vector contains an sgRNA expression frame and a Cas9 nuclease expression frame, wherein the sgRNA expression frame contains the target sequence as described above.
(3) introducing into a target plant cell the binary expression vector MtCRISPR/Cas9::MtDMP8MtDMP9, so that the sgRNA expression frame and the Cas9 nuclease expression frame are co-expressed in the target plant cells, and cutting the target fragments of the double-stranded MtDMP8 and MtDMP9 genes to induce the DNA repair function of the target plant cells, and randomly inserting or deleting bases at the target site to cause a shifting mutation, and then realizing the functional deletion mutation of the MtDMP8 and MtDMP9 genes in the cells.
(4) regenerating a plant by using the cell with the function deletion mutation of the MtDMP8 and MtDMP9 genes obtained in step (3).
(5) performing PCR amplification on the DNA segments containing the target sequence mentioned above in the MtDMP8 and MtDMP9 genes in the regenerated plants obtained in step (4), and sequencing.
(6) selecting the regenerated plants with functional deletion mutations at both alleles for phenotypic identification.

Wherein the functional deletion mutation refers to the occurrence of a terminator or reading frame shift at the target site in the natural encoding sequences for MtDMP8 and MtDMP9.

The Cas9 nuclease expression frame is located in the same vector containing the sgRNA expression frame.

In step (3), the binary expression vectors MtCRISPR/Cas9::MtDMP8, MtCRISPR/Cas9::MtDMP9 and MtCRISPR/Cas9::MtDMP8 MtDMP9 are introduced into the target graft cells so that the cells contain both the sgRNA and the Cas9 nuclease, of the target fragment of step. Under the joint action of the sgRNA and the Cas9 nuclease, the double-stranded target fragments of the MtDMP8 and MtDMP9 genes are cut, and then through the DNA repair function of the target plant cell itself, the random insertion and/or random deletion of the target fragments of MtDMP8 and MtDMP9 genes are finally realized in the cells.

The method of introducing the recombinant vector into the target plant cell is Agrobacterium-mediated stable transformation of callus. It is because the Agrobacterium-mediated method is utilized in the process of introducing the obtained recombinant vector into the cell of the target plant, the recombinant vector is introduced into the genetic DNA of the target plant, the fragment of the genetic DNA of the target plant is cut when cutting is performed.

In the present invention, the method for regenerating a plant is to obtain a plant by tissue culture of cells or tissues.

In step (5), the DNA fragments of the MtDMP8 and MtDMP9 genes containing the target fragments in the regenerated plant may be cloned by genomic PCR, and the targets of the amplified products are subjected to next generation sequencing. The method genomic PCR comprises the following steps of: designing site-specific primers for the genomic region containing the target fragment, and amplifying the genomic region containing the target fragment by taking genomic DNA of a regenerated plant as a template.

In a sixth aspect, the present invention claims application of the method described in the fifth aspect described above in plant haploid breeding.

In a seventh aspect, the present invention claims a plant haploid induction line constructed using the method described in the preceding fifth aspect.

In all of the above aspects, the plant may be a leguminous plant.

Further, the plant may be an alfalfa plant.

In a specific embodiment of the present invention, the plant is specifically Medicago truncatula, and more specifically, Medicago truncatula R108. Correspondingly, the hybrid offspring mentioned above is the hybrid offspring of the positive plant obtained by knocking out the DMP8 and DMP9 genes in Medicago truncatula R108 and Medicago truncatula A17.

### BRIEF DESCRIPTION OF DRAWINGS

FIG. 1 shows the results of pollen staining of Medicago truncatula R108, the single mutant dmp8, the single mutant dpm9, and the double mutant dmp8 dmp9.

FIG. 2 shows the statistics of the number of seeds in each pod of Medicago truncatula R108, the single mutant dmp8, the single mutant dpm9, and the double mutant dmp8 dmp9. ** indicates a highly significant difference (P<0.01). The sample capacity of the pods is 30.

FIG. 3 shows the phenotype of haploid plants induced from the self-crossing offspring of the double mutant dmp8 dmp9.

FIG. 4 shows the phenotype of haploid plants of hybrid offspring of the double mutant dmp8 dmp9 and Medicago truncatula A17.

### DETAILED DESCRIPTION OF THE INVENTION

The following examples facilitate a better understanding of the present invention but do not limit it. Methods of the experiments in the following examples are conventional methods unless otherwise specified. The test materials used in the examples below, unless otherwise specified, were all commercially available from the conventional biochemical reagent store. The quantitative tests in the following examples were repeated three times and the results were averaged.

Medicago truncatula R108 was provided by The Nobel Foundation (Available at: https://www.nobelprize.org/the-nobel-prize-organisation/the-nobel-foundation/).

Agrobacterium tumefaciens AGL1 was provided by the Institute of Biotechnology, Chinese Academy of Agricultural Sciences (i.e., the Applicant's workplace) and is available to the public from the Applicant.

YEP liquid medium: 10 g of peptone, 10 g of yeast extract and 5 g of sodium chloride were dissolved in an appropriate amount of distilled water, followed by volume adjustment to 1 L with distilled water, and autoclaved at 121° C. for 15 min.

Liquid medium for callus induction: 100 mL of mother liquor of macroelements, 1 mL of mother liquor of trace elements, 1 mL of mother liquor of organic elements, 20 mL of mother liquor of iron salts, 100 mg of inositol, 30 g of sucrose, 4 mg of auxin, and 0.5 mg of cytokinin were dissolved in an appropriate amount of distilled water, followed by volume adjustment to 1 L with distilled water, adjusted to pH 5.8, and autoclaved at 121° C. for 15 min.

Solid medium for callus induction: 100 mL of mother liquor of macroelements, 1 mL of mother liquor of trace elements, 1 mL of mother liquor of organic elements, 20 mL of mother liquor of iron salts, 100 mg of inositol, 30 g of sucrose, 4 mg of auxin, 0.5 mg of cytokinin, 200 mg of cephalosporin, 250 mg of Timentin, 2 mg of kanamycin and 3.2 g of Phytagel were dissolved in an appropriate amount of distilled water, followed by volume adjustment to 1 L with distilled water, adjusted to pH 5.8, and autoclaved at 121° C. for 15 min.

Medium for cell differentiation: 100 mL of mother liquor of macroelements, 1 mL of mother liquor of trace elements, 1 mL of mother liquor of organic elements, 20 mL of mother liquor of iron salts, 100 mg of inositol, 20 g of sucrose, 200 mg of cephalosporin, 250 mg of Timentin, 2 mg of kanamycin and 3.2 g of Phytagel were dissolved in an appropriate amount of distilled water, followed by volume adjustment to 1 L with distilled water, adjusted to pH 5.8, and autoclaved at 121° C. for 15 min.

Medium for rooting: 2.215 g of Murashige&Skoog Basal Medium with Vitamins (a product available from PhytoTechnology Laboratories, catalog number of 16B0519138A) was dissolved in an appropriate amount of distilled water, followed by volume adjustment to 1 L with distilled water, adjusted to pH 5.8, and autoclaved at 121° C. for 15 min.

Mother liquor of iron salts: 37.3 mg of disodium ethylenediamine tetraacetic acid and 27.8 mg of ferrous sulfate heptahydrate were dissolved in an appropriate amount of distilled water, followed by volume adjustment to 1 L with distilled water.

Mother liquor of macroelements: 1.85 g of magnesium sulfate heptahydrate, 28.3 g of potassium nitrate, 4.63 g of ammonium sulfate, 1.66 g of calcium chloride dihydrate and 4 g of potassium dihydrogen phosphate were dissolved in an appropriate amount of distilled water, followed by volume adjustment to 1 L with distilled water.

Mother liquor of trace elements: 1 g of manganese sulfate monohydrate, 500 mg of boric acid, 100 mg of zinc sulfate heptahydrate, 100 mg of potassium iodide, 10 mg of sodium molybdate dihydrate, 20 mg of copper sulfate pentahydrate and 10 mg of cobalt chloride hexahydrate were dissolved in an appropriate amount of distilled water, followed by volume adjustment to 1 L with distilled water.

Mother liquor of organic elements: 500 mg of nicotinic acid, 500 mg of thiamine hydrochloride and 500 mg of pyridoxine hydrochloride were dissolved in an appropriate amount of distilled water, followed by volume adjustment to 1 L with distilled water.

Formula of the Alexander staining solution: 5 mL of 95% ethanol, 500 μL of 1% malachite green, 2.5 mL of 1% acid fuchsin, 250 μL of 1% orange G, 12.5 mL of glycerol, and 2 mL of glacial acetic acid. The volume was then fixed to 50 mL with distilled water.

Formula of the LB01 buffer: 1M tris(hydroxymethyl) aminomethane (pH 7.5) 1.5 mL, 0.5M ethylened diamine tetra-acetic acid (pH 8.0), 8 mL of 1M potassium chloride, 400 μL of 5M sodium chloride, 5 mM spermine tetrahydrochloride, 200 μL of β-mercaptoethanol, and 100 μL of polyethylene glycol octyl phenyl ether.

### Example 1. Cloning of MtDMP8 and MtDMP9 Genes

### I. Cloning of MtDMP8 and MtDMP9 Genes

1. Total RNA of blooming flowers of Medicago truncatula R108 plant was extracted, which was reversely transcribed to obtain the cDNA of Medicago truncatula R108.
2. After completing the step 1, the cDNA of Medicago truncatula R108 was used as a template to amplify DMP8 and DMP9 genes respectively, wherein the primers for the amplification in the first round of DMP8 were DMP8-attB1-F and DMP8-attB2-R, and then the amplificated products from the first round of were used as templates, the primers attB adaptor-F and attB adaptor-R were used for the second round of amplification. DMP9 is amplified for one round by using primers of DMP9-attB1-F and DMP9-attB2-R. About 657 bp of PCR amplification products were obtained for both genes, and the PCR products were collected and subjected to BP reaction with vector pDONR207 to obtain two intermediate vectors.

DMP8-attB1-F:
(SEQ ID No. 9)
5′-caaaaaagcaggcttcATGGAACAAACACAACAAG-3′;
DMP8-attB2-R:
(SEQ ID No. 10)
5′-caagaaagctgggtcGGCAGACATGCATCCAAT-3′.
DMP9-attB1-F:
(SEQ ID No. 11)
5′-ggggacaagtttgtacaaaaaagcaggcttcATGGAACAAAC
TCAACAAG-3′;
DMP9-attB2-R:
(SEQ ID No. 12)
5′-ggggaccactttgtacaagaaagctgggtcGGAAGACATGCA
TCCAAT-3′.
attB adaptor-F:
(SEQ ID No. 13)
5′GTGGGGACAAGTTTGTACAAAAAAGCAGGCTTC-3′;
attB adaptor-R:
(SEQ ID No. 14)
5′GTGGGGACCACTTTGTACAAGAAAGCTGGGTC-3′.

3. Sequencing the intermediate vectors obtained in the step 2.

Sequencing results showed that the intermediate vector for the DMP8 gene contained the DNA molecule shown in SEQ ID No. 3. The DNA molecule shown in SEQ ID No. 3, i.e., the DMP8 gene, encodes DMP8 protein shown in SEQ ID No. 1. The intermediate vector for the DMP9 gene contains the DNA molecule shown in SEQ ID No. 4. The DNA molecule shown in SEQ ID No. 3, i.e., the DMP9 gene, encodes the DMP9 protein shown in SEQ ID No. 2.

### Example 2. Acquisition of to Generation MtDMP8MtDMP9 Gene Knockout Medicago truncatula

### I. Construction of MtCRISPR/Cas9::MtDMP8MtDMP9 Binary Vector

1. Selection of target sequences to target genes, two targets for each gene were designed respectively.

Among them, the two targets to the MtDMP8 gene were:

(SEQ ID No. 5)
5′-GCCACCACAAGAAGCCATGGGGG-3′;
(SEQ ID No. 6)
5′-TGGCCGTTCCTATAGATCGAAGG-3′

Two targets to the MtDMP9 gene were:

(SEQ ID No. 7)
5′-CCACCACAAGAGGCCATAGGCGG-3′;
(SEQ ID No. 8)
5′-TACCGATAGTTTTCACGGCGCGG-3′

2. pDIRECT-22C vector (Beijing Zhongyuan Ltd., Cat. No. of 91135-ADG) was used as a template, the following fragments were amplified using KOD DNA Polymerase:
Fragment 1: the primer combination used was CmYLCV+MtDMP8-B_gRNA1;
Fragment 2: the primer combination used was MtDMP8-C_gRNA1+MtDMP8-D_gRNA2;
Fragment 3: the primer combination used was MtDMP8-C_gRNA2+oCsy-E;
Fragment 4: the primer combination used was CmYLCV+MtDMP9-D2_gRNA3;
Fragment 5: the primer combination used was MtDMP9-C_gRNA3+MtDMP9-D_gRNA4;
Fragment 6: the primer combination used was MtDMP9-C_gRNA4+oCsy-E;
Fragment 7: the primer combination used was MtDMP8-C_gRNA2+MtDMP9-D_gRNA3.

The detailed information on each primer is as follows:

CmYLCV:
(SEQ ID No. 15)
5′-TGCTCTTCGCGCTGGCAGACATACTGTCCCAC-3′;
MtDMP8-B_gRNA1:
(SEQ ID No. 16)
5′-TCGTCTCCTCTTGTGGTGGCCTGCCTATACGGCAGTGAACCT
G-3′;
MtDMP8-C_gRNA1:
(SEQ ID No. 17)
5′-TCGTCTCAAAGAAGCCATGGGTTTTAGAGCTAGAAATAGC-3′;
MtDMP8-D_gRNA2:
(SEQ ID No. 18)
5′-TCGTCTCATAGGAACGGCCACTGCCTATACGGCAGTGAAC-3′;
MtDMP8-C_gRNA2:
(SEQ ID No. 19)
5′-TCGTCTCACCTATAGATCGAGTTTTAGAGCTAGAAATAGC-3′;
MtDMP9-D_gRNA3:
(SEQ ID No. 20)
5′-TCGTCTCACTCTTGTGGTGGCTGCCTATACGGCAGTGAAC-3′;
MtDMP9-CgRNA3:
(SEQ ID No. 21)
5′-TCGTCTCAAGAGGCCATAGGGTTTTAGAGCTAGAAATAGC-3′;
MtDMP9-D_gRNA4:
(SEQ ID No. 22)
5′-TCGTCTCAAAACTATCGGTACTGCCTATACGGCAGTGAAC-3′;
MtDMP9-C_gRNA4:
(SEQ ID No. 23)
5′-TCGTCTCAGTTTTCACGGCGGTTTTAGAGCTAGAAATAGC-3′;
MtDMP9-D2_gRNA3:
(SEQ ID No. 24)
5′-TCGTCTCACTCTTGTGGTGGCTGCCTATACGGCAGTGAACCT
G-3′;
oCsy-E:
(SEQ ID No. 25)
5′-TGCTCTTCTGACCTGCCTATACGGCAGTGAAC-3′.

3. The above seven fragments were recovered with a recovery kit, electrophoretically detect and measure the concentration. 5-7 ng of each fragment, 50 ng of the pDIRECT-22C vector, 0.5 μL of SapI enzyme, and 0.5 μL of Esp3I enzyme, 1 μL of T7 DNA Ligase, 10 μL of 2×T7 DNA Ligase buffer were added and mixed together, and ddH₂O was finally added to a volume of 20 μL. Among them, the fragments 1, 2 and 3 were required for the construction of the MtDMP8 single knockout mutant; the fragments 4, 5 and 6 were required for the construction of the MtDMP9 single knockout mutant; and the fragments 1, 2, 5, 6, and 7 were required for the construction of the double-knockout mutant of MtDMP8 MtDMP9.
4. The reaction procedure was as follows: 20×(37° C./5 min+25° C./10 min+4°) C HOLD, and if necessary, the number of ligation cycles may be increased.

The vectors that have been verified to be correct by sequencing were named MtCRISPR/Cas9::MtDMP8, MtCRISPR/Cas9::MtDMP9 and MtCRISPR/Cas9::MtDMP8MtDMP9.

### II. Acquisition of Recombinant Agrobacterium

The MtCRISPR/Cas9::MtDMP8MtDMP9 binary vector was introduced into Agrobacterium tumefaciens AGL1 to obtain a recombinant Agrobacterium, named AGL1/MtCRISPR/Cas9::MtDMP8MtDMP9.

The MtCRISPR/Cas9::MtDMP8 binary vector was introduced into Agrobacterium tumefaciens AGL1 to obtain a recombinant Agrobacterium, named AGL1/MtCRISPR/Cas9::MtDMP.

The MtCRISPR/Cas9::MtDMP9 binary vector was introduced into Agrobacterium tumefaciens AGL1 to obtain a recombinant Agrobacterium, named AGL1/MtCRISPR/Cas9::MtDMP9.

### III. Acquisition of to Generation MtDMP8 and MtDMP9 Gene Knockout Mutants

### 1. Preparation of a Solution for the Infection of Agrobacterium

(1) The single colonies of AGL1/MtCRISPR/Cas9::MtDMP8, AGL1/MtCRISPR/Cas9::MtDMP9 and AGL1/MtCRISPR/Cas9::MtDMP8MtDMP9 were inoculated into YEP liquid medium containing 50 mg/mL rifampicin and 50 mg/mL kanamycin, respectively, and incubated overnight at 28° C. and 200 rpm, to obtain culture medium 1.
(2) After completing step (1), 500 μL of culture medium 1 was inoculated into 5 mL of YEP liquid medium, and then 5 μL of acetosyringone aqueous solution at a concentration of 100 mg/mL was added for shaking culture at 28° C. and 200 rpm to obtain culture medium 2 with an OD₆₀₀ nm value of 0.8.
(3) After step (2) was completed, culture medium 2 was taken and centrifuged at 3800 rpm for 15 min to collect the bacterial cells.
(4) After step (3) was completed, the bacterial cells were taken and suspended in liquid culture medium containing 100 mg/L acetosyringone for induction of callus to obtain an infection solution with an OD₆₀₀ₙₘ value of 0.2.
   2. Acquisition of T₀ Generation MtDMP8 and MtDMP9 Gene Knockout Medicago truncatula
(1) Taking compound leaves of a Medicago truncatula R108 plant which grows for four weeks, and cutting 4-5 incisions into the leaves with a razor blade.
(2) After the step (1) is completed, placing the leaf small pieces in the infection solution obtained in the step 1, and shaking for 30 min under dark conditions.
(3) After the step (2) is completed, the leaf small pieces were transferred into solid culture medium for callus induction, and performing dark culture at 24° C. for 4 weeks (the culture medium was changed once every 2 weeks) to obtain the white embryogenic callus.
(4) After the step (3) is completed, the white embryogenic callus was transferred into the differentiation medium and cultured alternately under light and dark at 24° C. for 4 weeks (the medium was changed every 2 weeks) to differentiate into green embryoids.
(5) After the step (4) is completed, the green embryoid was transferred into a rooting medium, and cultured alternately under light and dark at 24° C. (the medium was changed once every two weeks), and the green embryoid was transfer to vermiculite after rooting and leafing until seedlings were formed.
(6) The genomic DNA of the obtained transgenic Medicago truncatula plants containing the recombinant vector having the MtDMP8 and MtDMP9 genes of Medicago truncatula targeted by CRISPR/Cas9 was extracted by CTAB, and the sequences containing the target regions were amplified by 2× Rapid Taq Master MixPCR using this DNA as a template and sent for sequencing.

Sequencing confirmed the availability of the single mutant dmp8, the single mutant dmp9, and the double mutant dmp8dmp9 (due to the insertion or deletion of nucleotides, the reading frame is shifted and the translated protein is terminated prematurely).

### IV. Phenotypic Analysis of MtDMP8 and MtDMP9 Knockout Mutants

1. Alexander staining of pollen and in vitro germination test were conducted on the mutants (single mutant dmp8, single mutant dmp9, and double mutant dmp8 dmp9) and wild-type (Medicago truncatula R108) obtained in step 3, respectively.

Alexander staining of pollen was performed as follows:
(1) The anthers of mature pollen but not yet pollinated flowers from Medicago truncatula were taken and placed into an appropriate amount of Carnot's fixative (anhydrous ethanol:glacial acetic acid=3:1) for 3-4 h at room temperature or overnight if necessary.
(2) The fixative was sucked out in a ventilated kitchen, and an appropriate volume of Alexander's staining solution was added and placed in an incubator at 37° C. for dark staining overnight.
(3) The stained anthers were transferred into a centrifuge tube containing 10% glycerol on the next day for decolorization for 45 min at room temperature. Finally, the staining of the pollen was observed under a microscope.

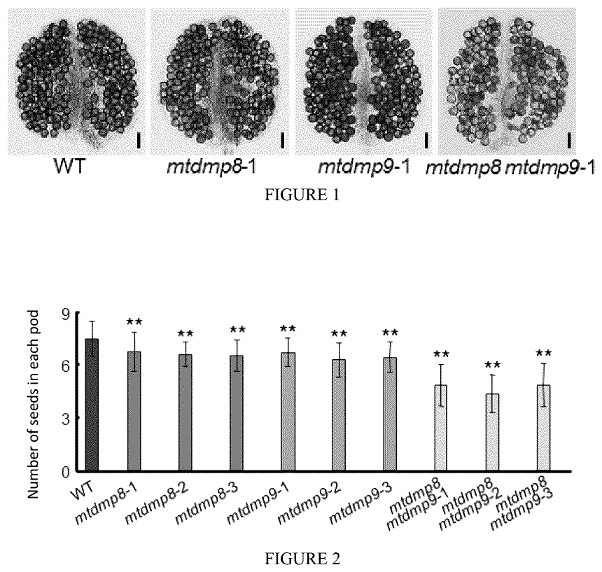

The results are shown in FIG. 1. As shown in the figure, pollen activity of dmp8 single mutant and dmp9 single mutant was not affected, but the pollen activity of the dmp8dmp9 double mutant was partially affected.
2. Statistics were performed on the number of seeds in each pod of the mutants (single mutant dmp8, single variant dmp9, and double mutant dmp8 dmp9) and wild-type (Medicago truncatula R108) obtained in step 3, and the results are shown in FIG. 2. It can be seen from the figure that the number of seeds in the pod of the mutant was reduced compared with that of the wild type, but the number of seeds in the pods of the dmp8dmp9 double mutant was reduced even more.
   V. Phenotypic Analysis of Haploid Plants from Self-Crossed Offspring

The self-crossed offsprings of the dmp8, dmp9, and dmp8 dmp9 mutants were analyzed by flow cytometry. The procedure for flow cytometry analysis of DNA content in the nucleus was as follows:
(1) The un-expanded compound leaves or just-expanded leaves of Medicago truncatula were put into 1 mL of LB01 buffer, and the leaves were cut with a new sharp blade for 2-3 min.
(2) The homogenate obtained in the first step was filtered through a 70 m filter membrane and added into a 1.5 mL centrifuge tube, and the lysed nuclei were collected by centrifugation at 135 g for 5 min.
(3) The supernatant was discarded, and 450 μL of LB01 buffer was added to re-suspend the precipitate. Then 25 μL of 1 mg/mL propidium iodide (PI) was added and stained on ice in the dark environment for 10 min.
(4) The stained sample was analyzed for DNA content in the nucleus by flow cytometry.

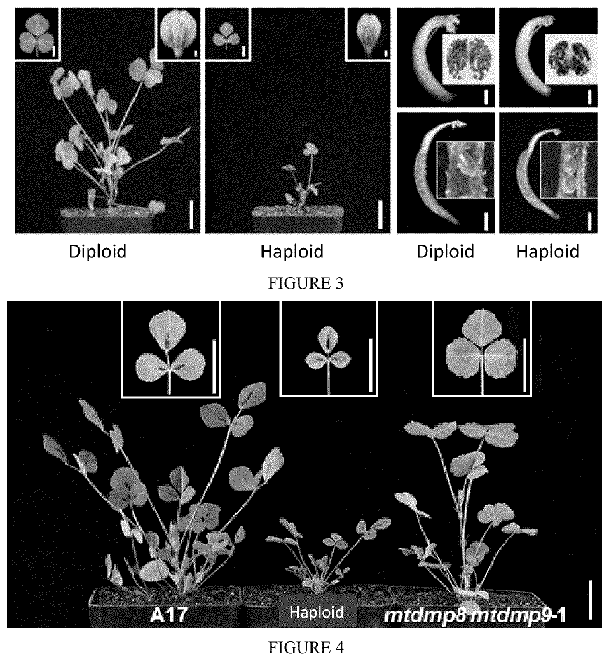

The results showed that no haploid was observed in the self-crossed offsprings of the single mutant dmp8, the single mutant dmp9, and the wild-type R108. In contrast, haploid plants were obtained from the self-crossing offsprings of the double mutant dmp8 dmp9, which were then subjected to phenotypic analysis, and the results were shown in FIG. 3.

VI. Phenotype Analysis of the Haploid from Hybridized Offsprings

The dmp8 dmp9 double mutant was hybridized with Medicago truncatula A17, and the hybridized offsprings were analyzed by flow cytometry. The results showed that the it may induce the haploid material of maternal origin by crossing the dmp8 dmp9 double mutant with their parents of different ecotypes of Medicago truncatula, and the haploid plant growth was consistent with the leaf phenotype of A17 plant. The results were shown in FIG. 4.

### INDUSTRIAL APPLICATIONS

By the method of designing the sgRNAs specifically targeting the genes encoding DMP8 and DMP9 of the Medicago truncatula, followed by utilizing the CRISPR-Cas9 system for knocking out the genes of DMP8 and DMP9 of the Medicago truncatula, a haploid induction system of the Medicago truncatula was created in the present invention. The present invention has great significance for haploid breeding of leguminous plants, and can effectively shorten the breeding period.

What is claimed is:

1. A set of proteins, consisting of protein A and protein B;
the protein A is any one of the followings:
(A1) a protein having an amino acid sequence of SEQ ID No. 1;
(A2) a protein obtained by substituting and/or deleting and/or adding of one or more amino acid residues to the amino acid sequence shown in SEQ ID No. 1 and having the same function as the protein of (A1);
(A3) a protein having 99% or more, 95% or more, 90% or more, 85% or more or 80% or more identity with the amino acid sequence defined in any one of (A1) to (A2) and having the same function as the protein of (A1) to (A2); and
(A4) a fusion protein obtained by attaching a protein tag to the N-terminus and/or C-terminus of the protein defined in any one of (A1)-(A3);
the protein B is any one of the followings:
(B1) a protein having an amino acid sequence of SEQ ID No. 2;
(B2) a protein obtained by substitution and/or deletion and/or addition of one or more amino acid residues to the amino acid sequence shown in SEQ ID No. 2 and having the same function as the protein of (B1);
(B3) a protein having 99% or more, 95% or more, 90% or more, 85% or more or 80% or more identity with the amino acid sequence defined in any one of (B1) to
(B2) and having the same function as the protein of (B1) to (B2); and
(B4) a fusion protein obtained by attaching a protein tag to the N-terminus and/or C-terminus of the protein defined in any one of (B1) to (B3).

2. A set of nucleic acid molecules, consisting of nucleic acid molecule A and nucleic acid molecule B;
the nucleic acid molecule A is a nucleic acid molecule capable of expressing the protein A of claim 1; the nucleic acid molecule B is a nucleic acid molecule capable of expressing the protein B of claim 1.

3. The set of nucleic acid molecules according to claim 2, wherein the nucleic acid molecule A is any one of the following DNA molecules:
(a1) a DNA molecule shown in SEQ ID No. 3;
(a2) a DNA molecule that hybridizes to the DNA molecule defined in (a1) under stringent conditions and encodes the protein A; and
(a3) a DNA molecule having 99% or more, 95% or more, 90% or more, 85% or more or 80% or more identity with the DNA sequence defined in (a1) or (a2) and encoding the protein A;
the nucleic acid molecule B is any one of the following DNA molecules:
(b1) a DNA molecule shown in SEQ ID No. 4;
(b2) a DNA molecule that hybridizes to the DNA molecule defined in (b1) under stringent conditions and encodes the protein B; and
(b3) a DNA molecule having 99% or more, 95% or more, 90% or more, 85% or more or 80% or more identity with the DNA sequence defined in (b1) or (b2) and encoding the protein B.

4. Any of the following biological materials:
P1. a set of expression cassettes consisting of an expression cassette A and an expression cassette B; wherein the expression cassette A is an expression cassette comprising the nucleic acid molecule A of claim 2; the expression cassette B is an expression cassette comprising the nucleic acid molecule B of claim 2;
P2. a set of recombinant vectors, consisting of recombinant vector A and recombinant vector B; wherein the recombinant vector A is a recombinant vector comprising the nucleic acid molecule A of claim 2; the recombinant vector B is a recombinant vector comprising the nucleic acid molecule B of claim 2;
P3. a set of recombinant bacteria, consisting of recombinant bacteria A and B;
wherein the recombinant bacterium A is a recombinant bacterium comprising the nucleic acid molecule A of claim 2; the recombinant bacterium B is a recombinant bacterium comprising the nucleic acid molecule B of claim 2;
P4. a set of transgenic cell lines, consisting of a transgenic cell line A and a transgenic cell line B; wherein the transgenic cell line A is a transgenic cell line comprising the nucleic acid molecule A of claim 2; the transgenic cell line B is a transgenic cell line comprising the nucleic acid molecule B of claim 2;
P5. a set of sgRNAs, consisting of sgRNA molecule A and sgRNA molecule B; wherein the sgRNA molecule A is an sgRNA molecule for target knockout of the nucleic acid molecule A of claim 2; the sgRNA molecule B is an sgRNA molecule for target knockout of the nucleic acid molecule B of claim 2;
P6. a set of CRISPR-Cas9 system, consisting of CRISPR-Cas9 system A and CRISPR-Cas9 system B; wherein the CRISPR-Cas9 system A consists of the sgRNA molecule A and the Cas9 protein in P5; and the CRISPR-Cas9 system B consists of the sgRNA molecule B and the Cas9 protein in P5; and
P7. CRISPR-Cas9 knockout vector, comprising the encoding genes of the sgRNA molecule A, the sgRNA molecule B and the Cas9 protein in P5.

5. A method for constructing a plant haploid induction line using any of the following:
(1) the set of proteins of claim 1;
(2) the set of nucleic acid molecules;
(3) the biological material.

6. A method of plant haploid breeding using any of the following:
(1) the set of proteins of claim 1;
(2) the set of nucleic acid molecules;
(3) the biological material.

7. The method according to claim 5, wherein the plant is a leguminous plant.

8. The method according to claim 6, wherein the plant is a leguminous plant.

9. The method according to claim 7, wherein the plant is an alfalfa plant.

10. The method according to claim 9, wherein the plant is Medicago truncatula.

11. A method for constructing a plant haploid induction line, characterized by comprising the steps of: reducing the expression and/or activity of both the protein A and the protein B of claim 1 in a recipient plant, and then obtaining the haploid induction line from self-crossed or hybridized offsprings.

12. The method according to claim 11, characterized by comprising the steps of: depressing expression of both the nucleic acid molecule A and the nucleic acid molecule B in the recipient plant to obtain a transgenic plant; and obtaining a haploid induction line from self-crossed or hybridized offsprings of the transgenic plant.

13. The method according to claim 12, wherein the nucleic acid molecule A and the nucleic acid molecule B in the recipient plant were both knocked out by CRISPR-Cas9 technology.

14. The method according to claim 13, which is carried out by introducing into the recipient plant the set of CRISPR-Cas9 system in P6 or the set of CRISPR-Cas9 knockout vector in P7.

15. The method according to claim 11, wherein, the plant is a leguminous plant.

16. The method according to claim 15, wherein, the plant is an alfalfa plant.

17. The method according to claim 16, wherein, the plant is Medicago truncatula.

18. A method of plant haploid breeding, using the method according to claim 11.

19. A plant haploid induction line constructed by the method of claim 11.
